(19) United States
(12) Patent Application Publication
(10) Pub. No.: US 2016/0166646 A1
(43) Pub. Date: Jun. 16, 2016

(54) METHODS AND PHARMACEUTICAL COMPOSITIONS FOR THE TREATMENT OF ACUTE EXACERBATIONS OF CHRONIC OBSTRUCTIVE PULMONARY DISEASE

(71) Applicants: INSERM (Institute National de la Santé et de la Recherche Médicale), Paris (FR); INSTITUTE PASTEUR DE LILLE, Lille (FR); CENTRE NATIONAL DE LA RECHERCHE SCIENTFIQUE (CNRS), Paris (FR); Université de Lille 1 Sciences et Technologies, Villeneuve d'Ascq (FR); Université de Droit et de la Santé de Lille 2, Lille (FR)

(72) Inventors: Philippe GOSSET, Lille (FR); Muriel PICHAVANT, Lille (FR); Riti SHARAN, Lille (FR)

(21) Appl. No.: 14/900,204

(22) PCT Filed: Jun. 27, 2014
(86) PCT No.: PCT/EP2014/063782

§ 371 Date: Dec. 21, 2015

(30) Foreign Application Priority Data
Jun. 28, 2013 (EP) 13305916.2

(51) Int. Cl. A61K 38/20; A61K 9/00; A61K 39/09; A61K 45/06

(52) U.S. Cl. CPC A61K 38/20; A61K 45/06; A61K 9/007; A61K 39/092; A61K 48/00

(57) ABSTRACT

The present invention relates to methods and pharmaceutical compositions for the treatment of acute exacerbation of chronic obstructive pulmonary disease. In particular, the invention relates to relates to a polypeptide selected from the group consisting of IL-22 polypeptides or IL-17 polypeptides for use in a method for the treatment of acute exacerbation of chronic obstructive pulmonary disease in a subject in need thereof.

### FIELD OF THE INVENTION

The present invention relates to methods and pharmaceutical compositions for the treatment of acute exacerbation of chronic obstructive pulmonary disease.

### BACKGROUND OF THE INVENTION

Chronic obstructive pulmonary disease (COPD) represents a severe and increasing global health problem. By 2020, COPD will have increased from 6th (as it is currently) to the 3rd most common cause of death worldwide. In the United States, COPD is believed to account for up to 120,000 deaths per year. The clinical course of COPD is characterized by chronic disability, with intermittent, acute exacerbations which may be triggered by a variety of stimuli including exposure to pathogens, inhaled irritants (e.g., cigarette smoke), allergens, or pollutants. “Acute exacerbation” refers to worsening of a subject's COPD symptoms from his or her usual state that is beyond normal day-to-day variations, and is acute in onset. Acute exacerbations of COPD greatly affect the health and quality of life of subjects with COPD. Acute exacerbation of COPD is a key driver of the associated substantial socioeconomic costs of the disease. Multiple studies have also shown that prior exacerbation is an independent risk factor for future hospitalization for COPD. In conclusion, exacerbations of COPD are of major importance in terms of their prolonged detrimental effect on subjects, the acceleration in disease progression and the high healthcare costs. However up to now there is no method for the treatment of acute exacerbation of COPD.

### SUMMARY OF THE INVENTION

The present invention relates to methods and pharmaceutical compositions for the treatment of acute exacerbation of chronic obstructive pulmonary disease.

### DETAILED DESCRIPTION OF THE INVENTION

Acute episodes of bacterial exacerbations mark the progression of chronic obstructive pulmonary disorder (COPD). These exacerbations often result in an increased inflammation of the respiratory tract causing death in many cases. Streptococcus pneumoniae (Sp) is one of the most commonly isolated bacteria during these episodes. Mechanisms responsible for the increased susceptibility to pathogens are unknown. The aim of the inventors was to characterize the immune response to Sp by using a mouse model of COPD. Mice were chronically exposed to cigarette smoke for 12 weeks and subsequently challenged with a sub-lethal dose of Sp. Systemic and local inflammation, immune responses, and bacterial burden were evaluated at 1, 3 and 7 days post-infection. Air mice were able to clear the bacteria within 24 hour post-infection, whereas COPD mice developed a strong lung infection. COPD mice show an increased bacterial load in their lung compartment as well as an increased inflammatory reaction. COPD mice show also a defect in immune cell recruitment (iNKT cells) and activation, and in IL-22 and IL-17 production in response to Sp. This was also confirmed in COPD patients compared to normal donors. Supplementation with recombinant IL-22 (or IL-17) in COPD mice before the challenge partially restored an efficient immune response to Sp. These data showed an increased susceptibility to Sp infection in COPD mice and identified IL-22 as a susceptibility factor in COPD exacerbation. Therefore targeting Th17 cytokines represent a potent strategy in COPD exacerbation.

Accordingly, the present invention relates to a polypeptide selected from the group consisting of IL-22 polypeptides or IL-17 polypeptides for use in a method for the treatment of acute exacerbation of chronic obstructive pulmonary disease in a subject in need thereof.

As used herein the term “acute exacerbation” has its general meaning in the art and refers to worsening of a subject's COPD symptoms from his or her usual state that is beyond normal day-to-day variations, and is acute in onset. Typically, the acute exacerbation of COPD is manifested by one or more symptoms selected from worsening dyspnea, increased sputum production, increased sputum purulence, change in color of sputum, increased coughing, upper airway symptoms including colds and sore throats, increased wheezing, chest tightness, reduced exercise tolerance, fatigue, fluid retention, and acute confusion, and said method comprises reducing the frequency, severity or duration of one or more of said symptoms. Acute exacerbation may have various etiologies, but typically may be caused by viral infections, bacterial infections, or air pollution. For example, approximately 50% of acute exacerbations are due primarily to the bacteria Streptococcus pneumoniae, Haemophilus influenzae, and Moraxella catarrhalis (all of them causing pneumonia). Viral pathogens associated with acute exacerbations in subjects with COPD include rhinoviruses, influenza, parainfluenza, coronavirus, adenovirus, and respiratory syncytial virus.

In some embodiments, the acute exacerbation of COPD is caused by a bacterial infection. In some embodiments, the acute exacerbation of COPD is caused by a viral infection. In some embodiments, the acute exacerbation of COPD is caused by air pollution.

In some embodiments, the subject experienced an acute exacerbation of COPD or is at risk of experiencing an acute exacerbation of COPD. In some embodiments, the subject has experienced at least one acute exacerbation of COPD in the past 24 months. In one particular embodiment, the subject has experienced at least one acute exacerbation of COPD in the past 12 months. In some embodiments, subject is a frequent exacerbator. As used herein the term “frequent exacerbator” refers to a subject who suffers from or is undergoing treatment for COPD and who experiences at least 2, and more typically 3 or more, acute exacerbations during a 12 month period.

In some embodiments, “treating” refers to treating an acute exacerbation of COPD, reducing the frequency, duration or severity of an acute exacerbation of COPD, treating one or more symptoms of acute exacerbation of COPD, reducing the frequency, duration or severity of one or more symptoms of an acute exacerbation of COPD, preventing the incidence of acute exacerbation of COPD, or preventing the incidence of one or more symptoms of acute exacerbation of COPD, in a human. The reduction in frequency, duration or severity is relative to the frequency, duration or seventy of an acute exacerbation or symptom in the same human not undergoing treatment according to the methods of the present invention. A reduction in frequency, duration or severity of acute exacerbation or one or more symptoms of acute exacerbation may be measured by clinical observation by an ordinarily skilled clinician with experience of treating COPD subjects or by subjective self evaluations by the subject undergoing treatment. Clinical observations by an ordinarily skilled clinician may include objective measures of lung function, as well as the frequency with which intervention is required to maintain the subject in his or her most stable condition, and the frequency of hospital admission and length of hospital stay required to maintain the subject in his or her most stable condition. Typically, subjective self evaluations by a subject are collected using industry-recognized and/or FDA-recognized subject reported outcome (PRO) tools. Such tools may allow the subject to evaluate specific symptoms or other subjective measures of quality of life. An example of one subject reported outcome tool is Exacerbations from Pulmonary Disease Tool (EXACT-PRO), which is currently being developed for evaluating clinical response in acute bacterial exacerbations by United BioSource Corporation along with a consortium of pharmaceuticai industry sponsors in consultation with the FDA.

In some embodiments, the treatment is a prophylactic treatment. As used herein, the term “prophylactic treatment” refer to any medical or public health procedure whose purpose is to prevent a disease. As used herein, the terms “prevent”, “prevention” and “preventing” refer to the reduction in the risk of acquiring or developing a given condition, or the reduction or inhibition of the recurrence or said condition in a subject who is not ill, but who has been or may be near a subject with the disease.

The term “IL-22 polypeptide” has its general meaning in the art and includes naturally occurring IL-22 and function conservative variants and modified forms thereof. The IL-22 can be from any source, but typically is a mammalian (e.g., human and non-human primate) IL-22, and more particularly a human IL-22. IL-22 consists of 179 amino acids. Dumoutier et al. reported for the first time the cloning of genes of murine and human IL-22 (Dumoutier, et al., JI, 164:1814-1819, 2000; U.S. Pat. Nos. 6,359,117 and 6,274,710). An exemplary amino acid sequence is provided by SEQ ID NO:1.

| SEQ ID NO: 1 (IL-22, Homo Sapiens): | |
|---|---|
| 1 | maalqksvss flmgtlatsc llllallvqg gaaapisshc rldksnfqqp yitnrtfmla |
| 61 | keasladnnt dvrligeklf hgvsmsercy lmkqvlnftl eevlfpqsdr fqpymqevvp |
| 121 | flarlsnrls tchiegddlh iqrnvqklkd tvkklgesge ikaigeldll fmslrnaci |

The term “IL-17 polypeptide” has its general meaning in the art and includes naturally occurring IL-17 and conservative function variants and modified forms thereof. IL-17 is a family of structurally related cytokines. Representative examples of IL-17 cytokines include, but are not limited to, IL-17/IL17A, IL-17B, IL-17C, IL-17D, and IL-17F. The IL-17 can be from any source, but typically is a mammalian (e.g., human and non-human primate) IL-17, and more particularly a human IL-17. In addition to the numerous literature references describing the sequence of IL-17, incorporated herein by reference in their entirety are the teachings provided in U.S. Pat. No. 6,043,344, which describes human, rat and herpes virus herpes IL-17 proteins and nucleic acid compositions. SEQ ID NO:1 and SEQ ID NO:2 from U.S. Pat. No. 6,043,344 are particularly incorporated herein by reference as being the teaching of methods of making variants of these sequences taught in that patent and the methods of testing the compositions in various assays described therein. Also incorporated herein by reference is U.S. Pat. No. 6,074,849. U.S. Pat. No. 6,569,645 also is incorporated herein by reference as providing a teaching of polypeptides homologous to IL-17 and nucleic acid molecules encoding those polypeptides. Other variants of IL-17 that may be useful in the present application include the IL-17E polypeptides and IL-17E-encoding nucleic acids that are described in U.S. Pat. No. 6,579,520. An exemplary amino acid sequence of IL17A is provided by SEQ ID NO:2:

| SEQ ID NO: 2 (IL-17A, Homo Sapiens): | |
|---|---|
| 1 | mtpgktslvs lllllsleai vkagitiprn pgcpnsedkn fprtvmvnln ihnrntntnp |
| 61 | krssdyynrs tspwnlhrne dperypsviw eakcrhlgci nadgnvdyhm nsvpiqqeil |
| 121 | vlrrepphcp nsfrlekilv svgctcvtpi vhhva |

“Function-conservative variants” are those in which a given amino acid residue in a polypeptide has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide which has at least 60% amino acid identity as determined by BLAST or FASTA algorithms, preferably at least 75%, most preferably at least 85%, and even more preferably at least 90%, and which has the same or substantially similar properties or functions as the native or parent protein to which it is compared.

In some embodiments, the IL-22 polypeptide has at least 60% of identity with SEQ ID NO:1

In some embodiments, the IL-17 polypeptide has at least 60% of identity with SEQ ID NO:2.

According to the invention a first amino acid sequence having at least 60% of identity with a second amino acid sequence means that the first sequence has 60; 61; 62; 63; 64; 65; 66; 67; 68; 69, 70; 71; 72; 73; 74; 75; 76; 77; 78; 79; 80; 81; 82; 83; 84; 85; 86; 87; 88; 89; 90; 91; 92; 93; 94; 95; 96; 97; 98; or 99% of identity with the second amino acid sequence.

In specific embodiments, it is contemplated that the polypeptides of the invention used in the therapeutic methods of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution.

A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain.

Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications.

In another particular embodiment the polypeptide of the invention is fused a Fc domain of an immunoglobulin. Suitable immunoglobins are IgG, IgM, IgA, IgD, and IgE. IgG and IgA are preferred IgGs are most preferred, e.g. an IgGl. Said Fc domain may be a complete Fc domain or a function-conservative variant thereof. The IL-17 polypeptide or IL-22 polypeptide of the invention may be linked to the Fc domain by a linker. The linker may consist of about 1 to 100, preferably 1 to 10 amino acid residues.

According to the invention, the polypeptide of the invention may be produced by conventional automated peptide synthesis methods or by recombinant expression. General principles for designing and making proteins are well known to those of skill in the art.

The polypeptides of the invention may be synthesized in solution or on a solid support in accordance with conventional techniques. Various automatic synthesizers are commercially available and can be used in accordance with known protocols as described in Stewart and Young; Tam et al., 1983; Merrifield, 1986 and Barany and Merrifield, Gross and Meienhofer, 1979. The polypeptides of the invention may also be synthesized by solid-phase technology employing an exemplary peptide synthesizer such as a Model 433A from Applied Biosystems Inc. The purity of any given protein; generated through automated peptide synthesis or through recombinant methods may be determined using reverse phase HPLC analysis. Chemical authenticity of each peptide may be established by any method well known to those of skill in the art.

As an alternative to automated peptide synthesis, recombinant DNA technology may be employed wherein a nucleotide sequence which encodes a protein of choice is inserted into an expression vector, transformed or transfected into an appropriate host cell and cultivated under conditions suitable for expression as described herein below. Recombinant methods are especially preferred for producing longer polypeptides.

A variety of expression vector/host systems may be utilized to contain and express the peptide or protein coding sequence. These include but are not limited to microorganisms such as bacteria transformed with recombinant bacteriophage, plasmid or cosmid DNA expression vectors; yeast transformed with yeast expression vectors (Giga-Hama et al., 1999); insect cell systems infected with virus expression vectors (e.g., baculovirus, see Ghosh et al., 2002); plant cell systems transfected with virus expression vectors (e.g., cauliflower mosaic virus, CaMV; tobacco mosaic virus, TMV) or transformed with bacterial expression vectors (e.g., Ti or pBR322 plasmid; see e.g., Babe et al., 2000); or animal cell systems. Those of skill in the art are aware of various techniques for optimizing mammalian expression of proteins, see e.g., Kaufman, 2000; Colosimo et al., 2000. Mammalian cells that are useful in recombinant protein productions include but are not limited to VERO cells, HeLa cells, Chinese hamster ovary (CHO) cell lines, COS cells (such as COS-7), W138, BHK, HepG2, 3T3, RIN, MDCK, A549, PC12, K562 and 293 cells. Exemplary protocols for the recombinant expression of the peptide substrates or fusion polypeptides in bacteria, yeast and other invertebrates are known to those of skill in the art and a briefly described herein below. Mammalian host systems for the expression of recombinant proteins also are well known to those of skill in the art. Host cell strains may be chosen for a particular ability to process the expressed protein or produce certain post-translation modifications that will be useful in providing protein activity. Such modifications of the polypeptide include, but are not limited to, acetylation, carboxylation, glycosylation, phosphorylation, lipidation and acylation. Post-translational processing which cleaves a “prepro” form of the protein may also be important for correct insertion, folding and/or function. Different host cells such as CHO, HeLa, MDCK, 293, WI38, and the like have specific cellular machinery and characteristic mechanisms for such post-translational activities and may be chosen to ensure the correct modification and processing of the introduced, foreign protein.

In the recombinant production of the polypeptides of the invention, it would be necessary to employ vectors comprising polynucleotide molecules for encoding the the polypeptides of the invention. Methods of preparing such vectors as well as producing host cells transformed with such vectors are well known to those skilled in the art. The polynucleotide molecules used in such an endeavor may be joined to a vector, which generally includes a selectable marker and an origin of replication, for propagation in a host. These elements of the expression constructs are well known to those of skill in the art. Generally, the expression vectors include DNA encoding the given protein being operably linked to suitable transcriptional or translational regulatory sequences, such as those derived from a mammalian, microbial, viral, or insect genes. Examples of regulatory sequences include transcriptional promoters, operators, or enhancers, mRNA ribosomal binding sites, and appropriate sequences which control transcription and translation.

The terms “expression vector,” “expression construct” or “expression cassette” are used interchangeably throughout this specification and are meant to include any type of genetic construct containing a nucleic acid coding for a gene product in which part or all of the nucleic acid encoding sequence is capable of being transcribed.

The choice of a suitable expression vector for expression of the peptides or polypeptides of the invention will of course depend upon the specific host cell to be used, and is within the skill of the ordinary artisan.

Expression requires that appropriate signals be provided in the vectors, such as enhancers/promoters from both viral and mammalian sources that may be used to drive expression of the nucleic acids of interest in host cells. Usually, the nucleic acid being expressed is under transcriptional control of a promoter. A “promoter” refers to a DNA sequence recognized by the synthetic machinery of the cell, or introduced synthetic machinery, required to initiate the specific transcription of a gene. Nucleotide sequences are operably linked when the regulatory sequence functionally relates to the DNA encoding the protein of interest (e.g., IL-17, IL-22, a variant and the like). Thus, a promoter nucleotide sequence is operably linked to a given DNA sequence if the promoter nucleotide sequence directs the transcription of the sequence.

Another aspect of the invention relates to a nucleic acid molecule encoding for a polypeptide of the invention (i.e. a IL-22 polypeptide or a Il-17 polypeptide) for use in a method for the treatment of acute exacerbation of COPD in a subject in need thereof.

Typically, said nucleic acid is a DNA or RNA molecule, which may be included in any suitable vector, such as a plasmid, cosmid, episome, artificial chromosome, phage or a viral vector as above described. So, a further object of the invention relates to a vector comprising a nucleic acid encoding for a polypeptide of the invention for use in a method for the treatment of acute exacerbation of COPD in a subject in need thereof.

By a “therapeutically effective amount” is meant a sufficient amount of the polypeptide (or the nucleic acid encoding for the polypeptide) to prevent for use in a method for the treatment of acute exacerbation of COPD at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The polypeptides of the invention (or the nucleic acid encoding thereof) may be combined with pharmaceutically acceptable excipients, and optionally sustained-release matrices, such as biodegradable polymers, to form pharmaceutical compositions.

“Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The pharmaceutical compositions may also be administered to the respiratory tract. The respiratory tract includes the upper airways, including the oropharynx and larynx, followed by the lower airways, which include the trachea followed by bifurcations into the bronchi and bronchioli. Pulmonary delivery compositions can be delivered by inhalation by the subject of a dispersion so that the active ingredient within the dispersion can reach the lung where it can, for example, be readily absorbed through the alveolar region directly into blood circulation. Pulmonary delivery can be achieved by different approaches, including the use of nebulized, aerosolized, micellular and dry powder-based formulations; administration by inhalation may be oral and/or nasal. Delivery can be achieved with liquid nebulizers, aerosol-based inhalers, and dry powder dispersion devices. Metered-dose devices are preferred. One of the benefits of using an atomizer or inhaler is that the potential for contamination is minimized because the devices are self contained. Dry powder dispersion devices, for example, deliver drugs that may be readily formulated as dry powders. A pharmaceutical composition of the invention may be stably stored as lyophilized or spray-dried powders by itself or in combination with suitable powder carriers. The delivery of a pharmaceutical composition of the invention for inhalation can be mediated by a dosing timing element which can include a timer, a dose counter, time measuring device, or a time indicator which when incorporated into the device enables dose tracking, compliance monitoring, and/or dose triggering to a subject during administration of the aerosol medicament. Examples of pharmaceutical devices for aerosol delivery include metered dose inhalers (MDIs), dry powder inhalers (DPIs), and air-jet nebulizers.

The polypeptide (or nucleic acid encoding thereof) may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In some embodiment, the polypeptide according to the invention is administered to the subject in combination with an anti-bacterial agent, such as antibiotics or antiviral agents. Suitable antibiotics that could be coadministered in combination with the polypeptide include, but are not limited to, at least one antibiotic selected from the group consisting of: ceftriaxone, cefotaxime, vancomycin, meropenem, cefepime, ceftazidime, cefuroxime, nafcillin, oxacillin, ampicillin, ticarcillin, ticarcillin/clavulinic acid (Timentin), ampicillin/sulbactam (Unasyn), azithromycin, trimethoprim-sulfamethoxazole, clindamycin, ciprofloxacin, levofloxacin, synercid, amoxicillin, amoxicillin/clavulinic acid (Augmentin), cefuroxime, trimethoprim/sulfamethoxazole, azithromycin, clindamycin, dicloxacillin, ciprofloxacin, levofloxacin, cefixime, cefpodoxime, loracarbef, cefadroxil, cefabutin, cefdinir, and cephradine. Example of antiviral agents include but are not limited to acyclovir, famciclovir, valaciclovir, ganciclovir, cidofovir; amantadine, rimantadine; ribavirin; zanamavir and/or oseltamavir; a protease inhibitor, such as indinavir, nelfinavir, ritonavir and/or saquinavir; a nucleoside reverse transcriptase inhibitor, such as didanosine, lamivudine, stavudine, zalcitabine, zidovudine; a non-nucleoside reverse transcriptase inhibitor, such as nevirapine, efavirenz.

Combination treatment may also include respiratory stimulants. Corticosteroids may be beneficial in acute exacerbations of COPD. Examples of corticosteroids that can be used in combination with the polypeptide (or the nucleic acid encoding thereof) are predniso lone, methylpredniso lone, dexamethasone, naflocort, deflazacort, halopredone acetate, budesonide, beclomethasone dipropionate, hydrocortisone, triamcinolone acetonide, fluocino lone acetonide, fluocinonide, clocortolone pivalate, methylprednisolone aceponate, dexamethasone palmitoate, tipredane, hydrocortisone aceponate, prednicarbate, alclometasone dipropionate, halometasone, methylprednisolone suleptanate, mometasone furoate, rimexo lone, predniso lone farnesylate, ciclesonide, deprodone propionate, fluticasone propionate, halobetasol propionate, loteprednol etabonate, betamethasone butyrate propionate, flunisolide, prednisone, dexamethasone sodium phosphate, triamcinolone, betamethasone 17-valerate, betamethasone, betamethasone dipropionate, hydrocortisone acetate, hydrocortisone sodium succinate, prednisolone sodium phosphate and hydrocortisone probutate. Particularly preferred corticosteroids under the present invention are: dexamethasone, budesonide, beclomethasone, triamcinolone, mometasone, ciclesonide, fluticasone, flunisolide, dexamethasone sodium phosphate and esters thereof as well as 6α,9α-difluoro-17α-[(2-furanylcarbonyl)oxy]-11β-hydroxy-16α-methyl-3-oxoandrosta-1,4-diene-17β-carbothioic acid (S)-fluoromethyl ester. Still more preferred corticosteroids under the present invention are: budesonide, beclomethasone dipropionate, mometasone furoate, ciclesonide, triamcino lone, triamcinolone acetonide, triamcinolone hexaacetonide and fluticasone propionate optionally in the form of their racemates, their enantiomers, their diastereomers and mixtures thereof, and optionally their pharmacologically-compatible acid addition salts. Even more preferred are budesonide, beclomethasone dipropionate, mometasone furoate, ciclesonide and fluticasone propionate. The most preferred corticosteroids of the present invention are budesonide and beclomethasone dipropionate.

Bronchodilator dosages may be increased during acute exacerbations to decrease acute bronchospasm. Examples of bronchodilators include but are not limited to β2-agonists (e.g. salbutamol, bitolterol mesylate, formoterol, isoproterenol, levalbuterol, metaproterenol, salmeterol, terbutaline, and fenoterol), anticholinergic (e.g. tiotropium or ipratropium), methylxanthined, and phosphodiesterase inhibitors.

In some embodiments, the polypeptide of the invention is administered to the subject in combination with a vaccine which contains an antigen or antigenic composition capable of eliciting an immune response against a virus or a bacterium. Typically, the vaccine composition is used to eliciting an immune response against at least one bacterium selected from the group consisting of Streptococcus pneumoniae, Staphylococcus aureus, Burkholderis ssp., Streptococcus agalactiae, Haemophilus influenzae, Haemophilus parainfluenzae, Klebsiella pneumoniae, Escherichia coli, Pseudomonas aeruginosa, Moraxella catarrhalis, Chlamydophila pneumoniae, Mycoplasma pneumoniae, Legionella pneumophila, Serratia marcescens, Mycobacterium tuberculosis, Bordetella pertussis. In particular, the vaccine composition is directed against Streptococcus pneumonia or Haemophilus influenza. More particularly, the vaccine composition is directed against Non-typeable Haemophilus influenzae (NTHi). Typically, vaccine composition typically contains whole killed or inactivated (eg., attenuated) bacteria isolate(s). However, soluble or particulate antigen comprising or consisting of outer cell membrane and/or surface antigens can be suitable as well, or instead of, whole killed organisms. In one or more embodiments, the outer cellular membrane fraction or membrane protein(s) of the selected isolate(s) is used. For instance, NTHi OMP P6 is a highly conserved 16-kDa lipoprotein (Nelson, 1988) which is a target of human bactericidal antibody and induces protection both in humans and in animal models. In chronic pulmonary obstructive disease (COPD), OMP P6 has been shown to evoke a lymphocyte proliferative response that is associated with relative protection from NTHi infection (Abe, 2002). Accordingly, OMP P6 or any other suitable outer membrane NTHi proteins, polypeptides (eg., P2, P4 and P26) or antigenic fragments of such proteins or polypeptides can find application for a NTHi vaccine. Soluble and/or particulate antigen can be prepared by disrupting killed or viable selected isolate(s). A fraction for use in the vaccine can then be prepared by centrifugation, filtration and/or other appropriate techniques known in the art. Any method which achieves the required level of cellular disruption can be employed including sonication or dissolution utilizing appropriate surfactants and agitation, and combination of such techniques. When sonication is employed, the isolate can be subjected to a number of sonication steps in order to obtain the required degree of cellular disruption or generation of soluble and/or particulate matter of a specific size or size range. In some embodiments, the vaccine composition comprises an adjuvant, in a particular TLR agonist. In one embodiment, the TLR agonist is selected from the group consisting of TLR1, TLR2, TLR3, TLR4, TLR5, TLR6, TLR7, TLR8, TLR9, TLR10, TLR11, TLR12, or TLR13 agonists.

In certain embodiments, oxygen requirements may increase and supplemental oxygen may be provided.

The invention will be further illustrated by the following figures and examples. However, these examples and figures should not be interpreted in any way as limiting the scope of the present invention.

### FIGURES

FIG. 1—COPD mice are more susceptible to Sp. Mice were chronically exposed to cigarette smoke over a period of 12 weeks and then intranasally challenged with 5×10⁴ or 5×10⁵ CFU of Streptococcus pneumoniae (Sp) or not (Mock). Survival of infected Air and infected COPD mice was monitored for a week (A). Inflammation was evaluated 1 day after Sp challenge (5×10⁴ CFU). Absolute numbers of neutrophils, lymphocytes and macrophages were analyzed in the BAL (B) and neutrophils in lung tissues (C) 24 h after infection. CFU counts were evaluated in the BAL, lung tissues and blood (D). Results were expressed as mean±SEM (n>10 per group).

FIG. 2—Concentrations of IFNγ, IL-17 and IL-22 failed to increase in response to Sp in COPD mice. Mice were chronically exposed to cigarette smoke over a period of 12 weeks and then intranasally challenged with 4×10⁴ CFU of Streptococcus pneumoniae (Sp) or not (Mock). IFNγ, IL-17 and IL-22 levels were evaluated in the BAL (A). Concentrations of Il-22 in the serum (B) and in supernatants from restimulated pulmonary cells (C) were measured 24 h after Sp challenge. Results were expressed as mean±SEM (n>10 per group).

FIG. 3—COPD mice exhibited a defect in their immune response to Sp. Mice were chronically exposed to cigarette smoke over a period of 12 weeks and then intranasally challenged with 5×10⁴ CFU of Streptococcus pneumoniae (Sp) or not (Mock). Immune cells were quantified in lung tissues, and their activation status (expression of CD69) was evaluated (A). Cytokine profile was evaluated in NK, NKT, Lin− and T cells by intracellular staining, among pulmonary CD45⁺ cells (B and C). We have reported representative dot blot of the selected sub-populations (B). The mean percentage of positive cells was calculated for each sub-populations (C). Results were expressed as mean±SEM. *: p<0.05 vs controls.

FIG. 4—Exogenous IL-22 improves the immune response of COPD mice to Sp. Mice were chronically exposed to cigarette smoke over a period of 12 weeks and then intranasally challenged with 5×10⁴ CFU of Streptococcus pneumoniae (Sp) or not (Mock). Recombinant IL-22 was intranasally given to mice the day before Sp infection. CFU counts were evaluated in BAL, lung tissues and Blood (A). Immune cells percentages were analyzed in lung tissues, as well as activation marker (CD69 in NKT cells and CD86 in alveolar macrophages and dendritic cells) expression (B). IL-17 and IFNγ levels were evaluated in supernatants from restimulated pulmonary cells collected 24 h after Sp challenge (C). Anti-microbial peptide mRNA levels were analyzed in lungs tissues 1 and 3 days post-infection (D). Results were expressed as mean±SEM. *: p<0.05 vs controls.

FIG. 5—COPD patients have a defective response to Sp. Production of IL-17, IL-22 and IFNγ was evaluated by ELISA in supernatants from mononuclear cells from not smoker healthy subjects (control), smokers healthy subjects and COPD patients. Results were expressed as mean±SEM. *: p<0.05 vs controls. In parallel, intracellular staining for IL-22 and IFNγ was performed in subpopulations of innate lymphocytes including NK and ILC.

### EXAMPLE 1

Material & Methods

Cigarette Smoke Exposure

Mice were exposed to CS generated from 5 cigarettes per day, 5 days a week, and up to 12 weeks using a smoke machine (Emka, Scireq, Canada).

Measurement of Lung Function

Lung function was assessed by invasive measurement, as previously described (21). Aerosolized methacholine (Sigma) was administered in increasing concentrations (from 2.5 to 160 mg/ml of methacholine). We computed airway resistance, dynamic compliance and lung elastance by fitting flow, volume and pressure to an equation of motion (Flexivent System, Scireq).

Cytokine Quantification

Mouse and human IL-2, IL-4, IL-17, IL-22 and IFN-γ concentrations were measured in supernatants of coculture by ELISA (R&D systems and e-Biosciences).

Reverse Transcriptase-Polymerase Chain Reaction (RT-PCR) Analysis

Quantitative RT-PCR was performed to quantify mRNA of interest (Table 1). Results were expressed as mean±SEM of folds (2^{-ΔΔCt}) of the gene expression using β-actin as a reference, and compared to controls (air) calculated for each experiment.

Bacterial Infection

Two strains of bacteria were used to exacerbate COPD: Streptococcus pneumoniae (Sp) serotype 1, and non typable Haemophilus influenza (NTHI). Bacteria stocks were kept frozen at −80° C. Bacteria were defrost just before the infection, and the number of cfu was determined on chocolate plates. Infection was performed by intranasal route (50 μl/mouse).

Results

Development of the Experimental Model for the Exacerbation of COPD

We first aimed at developing a mouse model mimicking features associated to COPD. For this purpose, mice were chronically exposed to the main stream of cigarette smoke: 5 cigarettes per day, 5 days a week, over a period of 12 weeks. 3R4F reference cigarettes were obtained from Kentucky University, USA, and were used for all our in vivo exposure to cigarette smoke. After 3 months of exposure, lung function, cellular infiltration and activation, as well as airway remodelling were evaluated. As a result, repeated exposure of C57BL/6 mice to CS induced an inflammatory lung reaction, mimicking COPD. This was characterized by neutrophil and macrophage recruitment, as early as one week post CS-exposure (data not shown). Mice chronically exposed to CS (called COPD mice) show a decline in their lung function as compared with mice exposed to air. Chronic exposure to cigarette smoke induced an increased airway resistance in response to methacholine and was also associated with a destruction of alveolar walls (emphysema). Alteration of lung function was associated with a lung inflammatory reaction characterized by recruitment of neutrophils, macrophages, dendritic cells (DC), natural killer (NK) and NKT cells. The migration of these inflammatory cells was associated with their activation in lung tissues (Pichavant et al, Mucosal Immunology, 2014, 7(3):568). We used these experimental settings to develop two exacerbation models of COPD: the first one using Streptococcus pneumoniae (Sp), and the second one with non typable Haemophilus influenza (NTHI).

Alteration of the Immune Response to S. pneumoniae in COPD Mice.

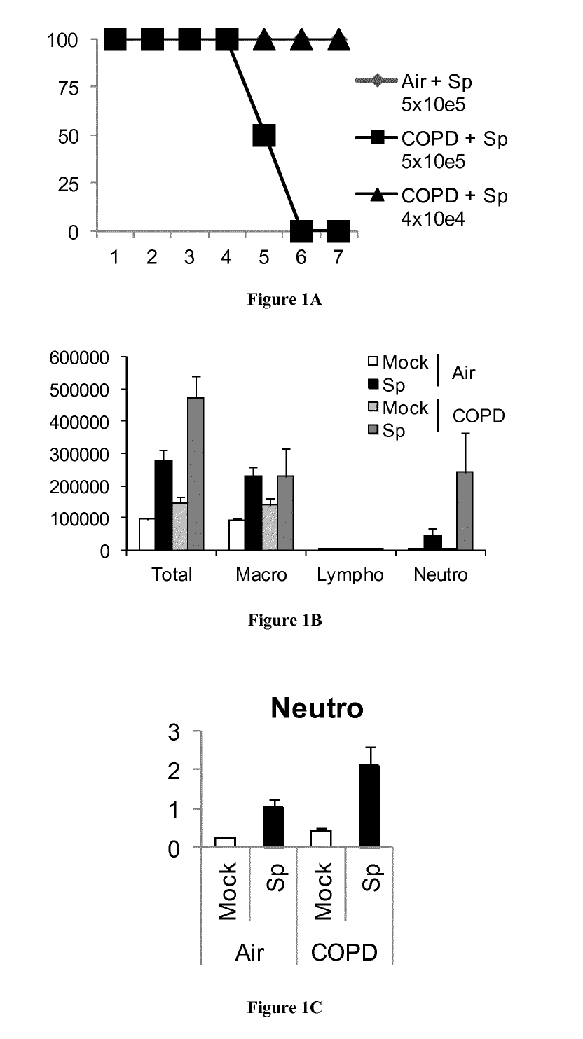

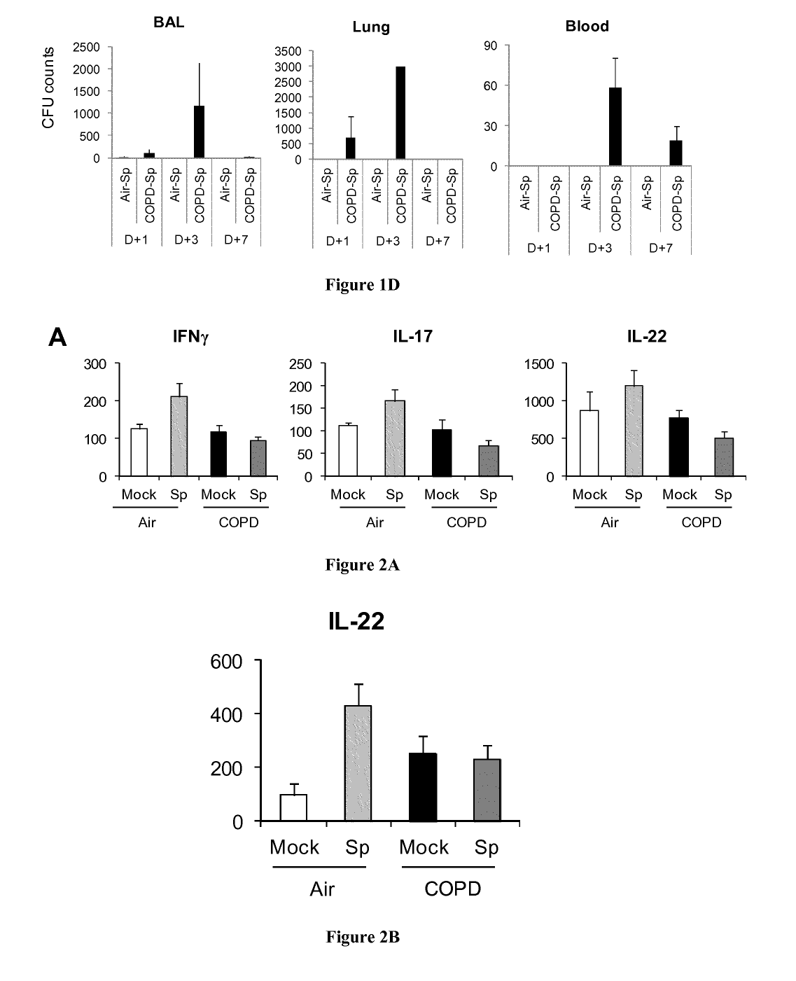

In the first set of experiments, we used Sp to exacerbate COPD. COPD mice were challenged intranasally with a sub-lethal dose of Sp (5×10⁵ CFU/mouse). Air mice survived after Sp challenge, whereas COPD mice died after exposure to the same dose of Sp, within 6 days (FIG. 1A). Our results demonstrated that COPD mice had a higher bacterial load in their bronchoalveolar lavage (BAL) than Air mice that cleared up all the bacteria within 24 hours post-infection. Lung inflammation was exacerbated in COPD mice. Indeed, COPD mice showed a higher percentage of neutrophils, associated to an increased number of total cells and neutrophils in the BAL. COPD mice exposed to Sp developed also a stronger inflammatory reaction in their lungs, characterized by neutrophil accumulation. In addition, NKT cell recruitment and activation (as shown as CD69 expression) failed in COPD mice after Sp challenge. This defect was also associated to a reduced maturation of DC after Sp.

This defect in innate immune cell recruitment and/or maturation after Sp challenge was associated to a defect in cytokine production. Sp significantly increased the levels of IL-22, IFN-γ, IL-17 in BAL fluids from air mice. In contrast, no changes were observed in COPD mice. The defect in IL-22 due to Sp challenge was also seen in lung cell supernatants, as well as in the serum. In contrast no difference was detected for IFN-γ, IL-4 and IL-17 in the lung cells and these cytokines are undetectable in the serum.

Since the first dose of Sp we used was lethal for COPD mice after 6 days, we repeated the experiments with a lower dose of 5×10⁴CFU/mouse. All COPD mice survived after challenge with a lower dose of Sp.

Air mice cleared up all the bacteria within 24 hours, whereas the clearance of Sp was delayed in COPD mice. The bacterial load was maximal 3 days post-infection, in the BAL, the lungs and the blood. 7 days post-infection, COPD mice almost cleared up the bacteria.

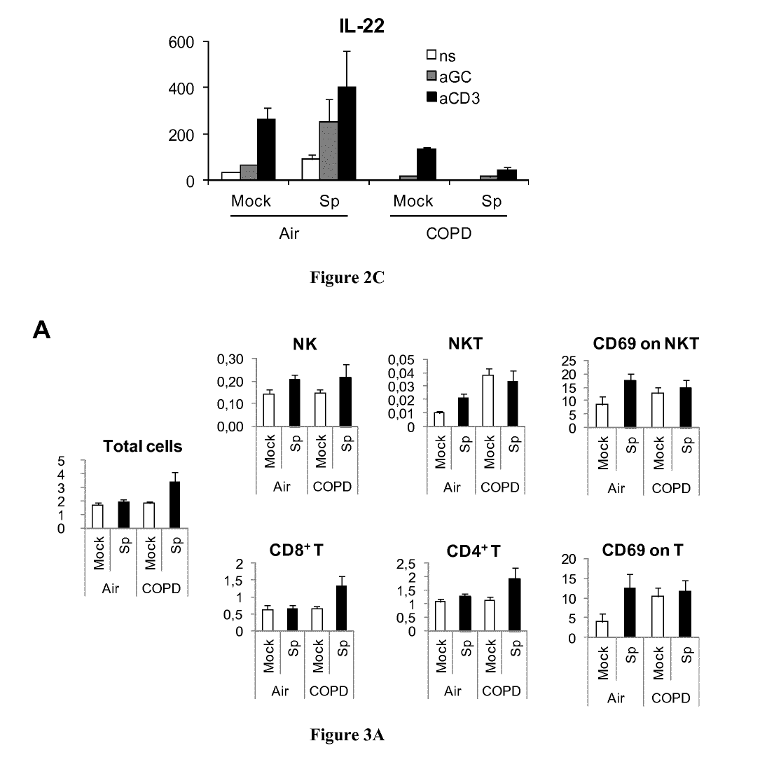

As described with the dose of 5×10⁵ CFU/mouse, the same immune trends were observed with 5×10⁴ CFU/mouse (FIG. 1B). Indeed, this dose induced an inflammation as seen in the increased total cell numbers in the BAL of COPD compared to air mice. A defect in IL-22 levels in BAL was also observed (FIG. 2A).

Since we observed a defect in IL-17 and IL-22 in response to Sp in COPD mice, we proposed Th17 cytokines as potential targets to restore an appropriate response to infection in COPD mice. In a first set of experiments, we administrated recombinant murine IL-22 to COPD mice 3 days and 6 hours before the challenge with the sublethal dose of Sp.

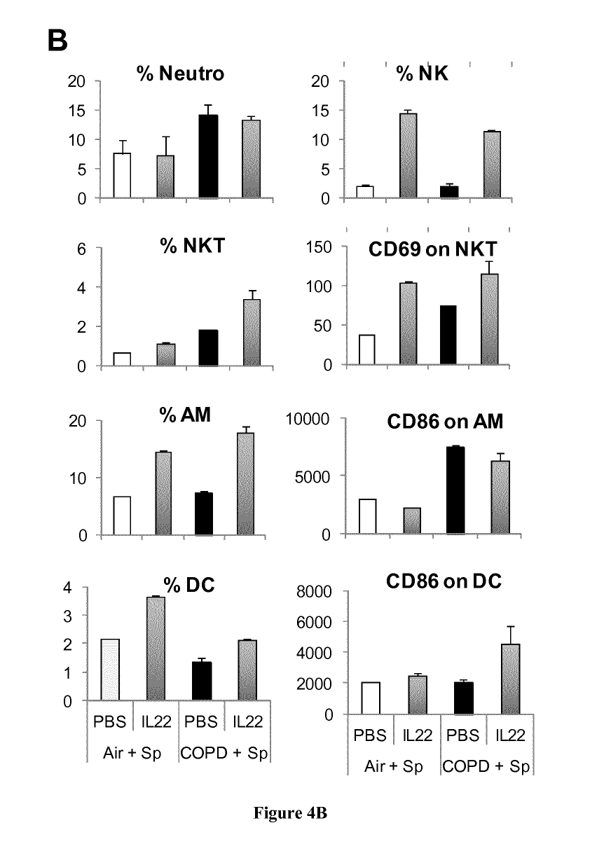

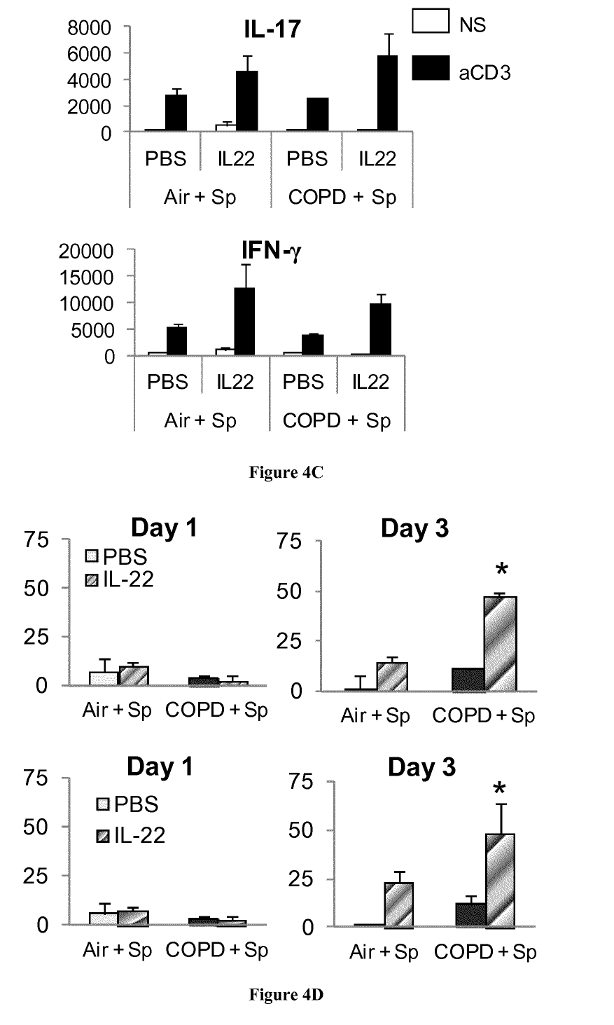

Supplementation with recombinant murine IL-22 prior Sp challenge decreases the amounts of CFU in BAL, lungs and blood. Exogenous IL-22 also increases the levels of anti-microbial peptides in the lung tissues (FIG. 4).

Alteration of the Immune Response to NTHI in COPD Mice.

In the second set of experiments, we used NTHI to exacerbate COPD. COPD mice were challenged intranasally with two sub-lethal dose of NTHI (5×10⁶ and 5×10⁷ CFU/mouse). All mice survived after NTHI challenge.

There was an increase in the bacterial load in lungs and BAL of COPD exposed infected mice as compared to air infected mice at day 2 post-infection with a marked increase in lungs and BAL of COPD mice infected with the higher dose of NTHI (5×10⁷ CFU). This is dependent on the administrated dose since we did not detect this increase at the lower dose.

In addition, we also observed an increase in the bacterial load in blood of infected COPD mice as compared to air infected mice at day 2 post-infection. Hence, this data confirms the susceptibility to NTHI infection in COPD mice as compared to air mice.

A higher level of IFN-γ, IL-1β, IL-6, IL-2, IL-17, IL-22 and TNF-α was observed in the BAL fluid and the lung lysates of air mice infected with the highest dose of NTHI as compared with not infected mice with a level positively related to the administrated dose of NTHI. The concentrations of IFN-γ, IL-1β, IL-6, IL-2, IL-17 and TNF-α were higher in the lung of infected COPD mice as compared to air mice both on day 1 and day 2 after infection. This increase was only significant at the highest dose of NTHI. In marked contrast, the levels of IL-22 were decreased with both the doses of NTHI in the BAL and lung lysates from COPD infected mice as compared to air mice. Although the decrease was present at day 1 and 2, the difference was more evident on day 2 as compared to day 1 and with the higher dose as compared to lower dose. Similarly, the secretion of IL-22 was also altered in in vitro restimulated lung cells from infected COPD mice as compared to infected air mice. This is specific to this cytokine since IL-17 and IFN-γ are enhanced in the same conditions.

In the serum, IFN-γ and IL-6 levels were observed to be higher in COPD infected mice as compared to air infected mice though no marked difference was observed in the cytokine levels between days 1 and 2 in both the doses. No detectable levels of IL-17, IL-22 and TNF-α were observed in the serum of mice infected with NTHI.

The total numbers of cells in the BAL and the lung were consistently higher in COPD mice infected with NTHI (5×10⁷ CFU) compared to infected air mice. In addition, the percentages of neutrophils were higher in BAL and among lung cells of COPD mice infected with NTHI (5×10⁷ CFU), compared to the air infected mice and the non infected COPD mice. There was a marked increase in the recruitment and activation status of DC in COPD mice as compared to air mice infected with the higher dose on day 2. Moreover, we reported increased percentages of iNKT cells, NK cells and T lymphocytes in COPD mice compared to air mice infected with the higher dose of NTHI.

The histopathological analysis revealed a marked increase in inflammation. A strong alveolar remodelling were observed on lung sections of COPD mice infected with NTHI (5×10⁷ CFU) as compared to air infected mice at day 2. Though the inflammation and the alveolitis were moderate in air infected mice, the alveolitis was still prominent in CS exposed mice with a strong thickening of the alveolar wall. This is indicative of a marked remodeling of lung tissue with the higher dose as compared to the infected Air-mice. In addition, alveolar remodelling (thickening of the alveolar wall) was still observed at day 7. Moreover, some inflammation still persisted in the COPD exposed and infected mice whereas it was clearly diminished in the controls.

Conclusion:

Overall, these data demonstrate that COPD features can be exacerbated by pathogens, such as Streptococcus pneumoniae and non typable Haemophilus influenzae. Our focus was to understand why COPD mice are more susceptible to infection than air mice, in order to propose some potential therapeutic aspects. As a result, our data showed that there is a defect in the production of IL-17 and IL-22, in COPD mice in response to the infection. Moreover, supplementation with recombinant mouse IL-22 allows the control of bacterial infection with S. pneumoniae in COPD mice. Therefore, restoring or compensating this defect, for example with recombinant Th17 cytokines, might represent one of the therapy to limit infection in COPD.

### EXAMPLE 2

Material and Methods

Patients with COPD

Peripheral blood were collected in stable COPD patients (n=10), in smokers (without COPD, n=12)) and in non smoker healthy controls (n=13) (CPP 2008-A00690-55) in order to evaluate ex vivo the Th17 response to infection with S. pneumoniae. Peripheral blood mononuclear cells (PBMC) were purified on Ficoll Paque gradient and 3×10⁶ cells/ml in complete RPMI1640 were exposed to S. pneumoniae (MOI=2) or to a positive control, phytohemagglutinin (1 μg/ml) (PHA, Difco). After 90 min, antibiotics were added to stop bacteria growth and supernatants were collected after 24 h incubation. In parallel, another batch of cells was incubated with brefeldin (10 μg/ml, Sigma Co) for 4 h before collection and was used for intracellular staining of cytokines

Mice

Six- to eight-week-old male wild-type (WT) C57BL/6 (H-2D^{b}) mice were purchased from Janvier (Le Genest-St-Isle, France). For S. pneumoniae infection, mice were maintained in a biosafety level 2 facility. All animal work conformed to the guidelines of Animal Care and Use Committee from Nord Pas-De-Calais (agreement no. AF 16/20090).

Reagents and Abs

α-GalCer was from Axxora Life Sciences (Coger S.A., Paris, France). mAbs against mouse CD3 (APC-conjugated), CD5 (FITC-conjugated), NK1.1 (PerCp-Cy5.5-conjugated), TCR-β (V450-conjugated), CD25 (APC-conjugated), CD69 (Alexa700-conjugated), CD11b (V450-conjugated), Ly-6G (APC-Cy7-conjugated), CD8 (V500-conjugated), CD4 (APC-conjugated), CD103 (PE-conjugated), CD11c (APC-conjugated), CD45 (Q-dot605-conjugated), F4/80 (PerCP-Cy5.5-conjugated), CD86 (PE-conjugated), CD40 (PE-conjugated), I-Ab (FITC-conjugated), CD11c (PE-Cy7-conjugated), F4/80 (PerCP-Cy5.5-conjugated), CD11b (V450-conjugated) and CD103 (PE-conjugated) and isotype controls were purchased from Biolegend (Le Pont de Claix, France) or BD Biosystems (Rungis, France). Anti-IL-22 (PE-conjugated) and -IL-17 (APC-conjugated) were also used for intracellular staining with the corresponding isotype controls (eBiosciences). mAb against human CD were also used including anti-CD11c, CD14, CD19, CD20 (PE-CF594-conjugated), CD117, -TCRγλ (V450-conjugated), -CD4, -CD3 (Alexa-700 conjugated), -CD8, -CD127 (V500 conjugated), -CD196-, -CD3 (BV605 conjugated) -CD25, -CD86 (APC-conjugated), -CD56, -Vα7.2 (PerCP-Cy5.5 conjugated), -TCR Vα24Jα18, -CD161 (PE-Cy7 conjugated) and CD45 (APC-H7 conjugated) (BD Biosciences, Biolegend and Myltenyi Biotech) as well as the Alexa488 anti-IFN-γ, Alexa647 anti-IL-17 (BD Biosciences) and PE anti-IL-22 antibodies (e-Biosciences) and the isotype controls. 3R4F research cigarettes were purchased from University of Kentucky. Recombinant murine IL22 (Myltenyi Biotech)

Streptoccus pneumoniae and Bacterial Counts

Mice were inoculated by the intranasal route with S. pneumoniae serotype 1 clinical isolate E1586 sequence type ST304 is described elsewhere (Munoz N, et al 2010; Zemlickova H, et al. 2005; Marques J M, et al. 2012). Mice were anesthetized and administered i.n. with 5×10⁴ bacteria. Mice were monitored daily for illness and mortality for 7 days. A morphology-based differential cell count was conducted on cytospin preparations from the bronchoalveolar lavage (BAL) fluid samples and stained with Diff-Quik solution (Sigma). Bacterial burden in the lungs, BAL and blood samples was measured by plating lung homogenates, BAL or blood samples onto blood agar plates. Colony-forming units were enumerated 24 hours later.

Assessment of Airway Inflammation and Remodeling

Mice were sacrificed for sampling the lung lumen by bronchoalveolar lavage (BAL). Total cell numbers per BAL was determined. A morphology-based differential cell count was conducted on cytospin preparations, after staining with Diff-Quik solution (Sigma). For histopathology, lungs were fixed by inflation and immersion in Immuno-HistoFix and embedded in Immuno-HistoWax. To evaluate airway inflammation, lung slices (4-μm sections) were done for H&E staining

Pulmonary cells from air or COPD mice were prepared as previously described (19) and were analyzed by flow cytometry. To analyze iNKT cell cytokine profile, pulmonary cell suspensions were incubated with phorbol 12-myristate 13-acetate (PMA; 20 ng/ml) and ionomycin (500 ng/ml) for 3 h. Cells were stained for the identification of innate and T lymphocytes and then fixed, permeabilized, and incubated with PE-conjugated mAb against IL-22 and APC-conjugated mAb against IL-17, or control rat IgG1 mAb in permeabilization buffer. Cells were acquired and analyzed on a Fortessa (Becton Dickinson, Rungis, France) cytometer, and using the FlowJo software respectively.

Cytokine production was analyzed in total lung cells. For this, 5×10⁵ lung cells were seeded on 96-well plates and then stimulated with α-GalCer (100 ng/ml) and coated anti-CD3 Ab. Forty-eight hours later, supernatants were collected and analyzed for IFN-γ, IL-22, and IL-17 concentration by ELISA (R&D Systems).

Results:

Intranasal Challenge with S. pneumoniae Exacerbates Lung Inflammation in COPD Mice

We first aimed to establish a mouse model of COPD exacerbation using Streptococcus pneumoniae (serotype 1) as the trigger. Whereas Air-mice survived after being challenged with 5×10⁵ CFU, all mice exposed to CS mice died within a week (FIG. 1A). Using a sub-lethal dose of 5×10⁴ CFU, COPD and air-mice survived. Inflammation due to Sp challenge is increased in COPD mice compared to Air mice, and was mainly characterized by the recruitment of neutrophils in the BAL (FIG. 1B) and the lungs (FIG. 1C). Air mice cleared the bacteria within 24 h, whereas bacterial load increased until day 3 in COPD mice. As shown in FIG. 1D, Sp persisted in the BAL, the lung compartment and the blood up to 7 days post-infection showing that bacterial clearance was delayed. These data suggest that COPD are more susceptible to Sp, exhibit a greater inflammation and a delayed clearance of Sp, compared to Air mice.

Th17 Cytokines, as Susceptibility Factors for COPD Exacerbation?

We next looked at the immune response by analyzing the cytokine profile in the BAL, lungs and sera. Challenge with a sub-lethal dose of Sp induced higher levels of IFN-γ, IL-17 and IL-22 in the BAL of air mice. In contrast, no increase in these cytokines was observed in COPD mice in response to Sp (FIG. 2A). The same profile for IL-22 was found in the serum (FIG. 2B), whereas IFN-γ and IL-17 were undetectable in all mice. Restimulated pulmonary cells, either with αGC, an iNKT cell agonist, or anti-CD3 Abs from infected COPD mice did not increased IL-22 production as compared with mock COPD mice whereas SP1-infected air mice significantly enhanced their levels (FIG. 2C). In comparison with air mice, IFN-γ response was stronger in COPD mice, and was increased by Sp, and no significant difference was observed for IL-17. No significant difference between air and COPD mice was seen at the mRNA levels of anti-microbial peptides such as Reg-3β, Reg-3γ and S100A9.

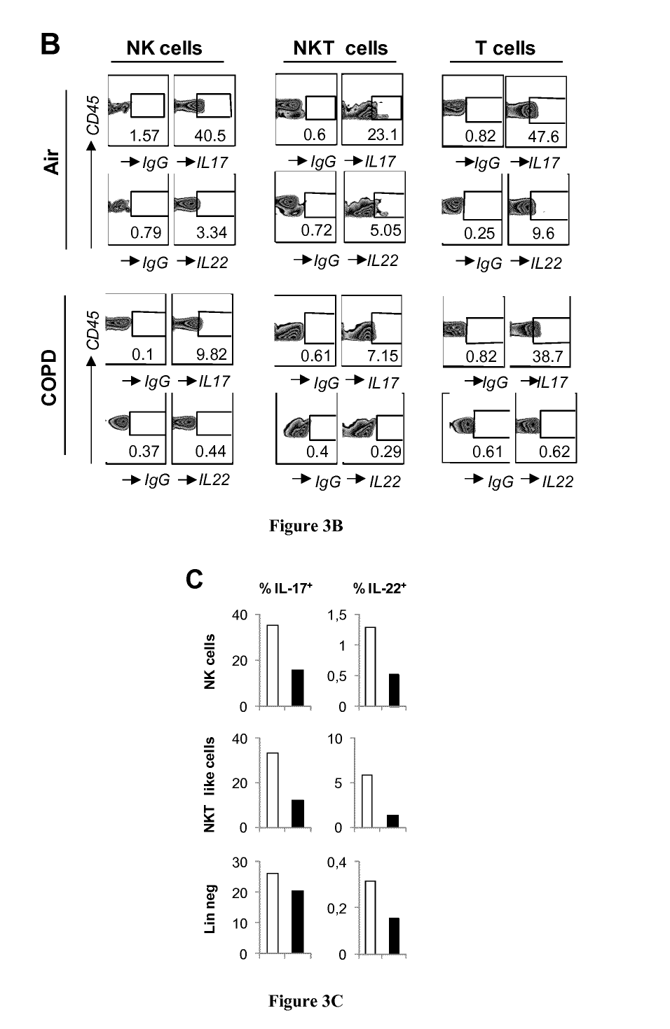

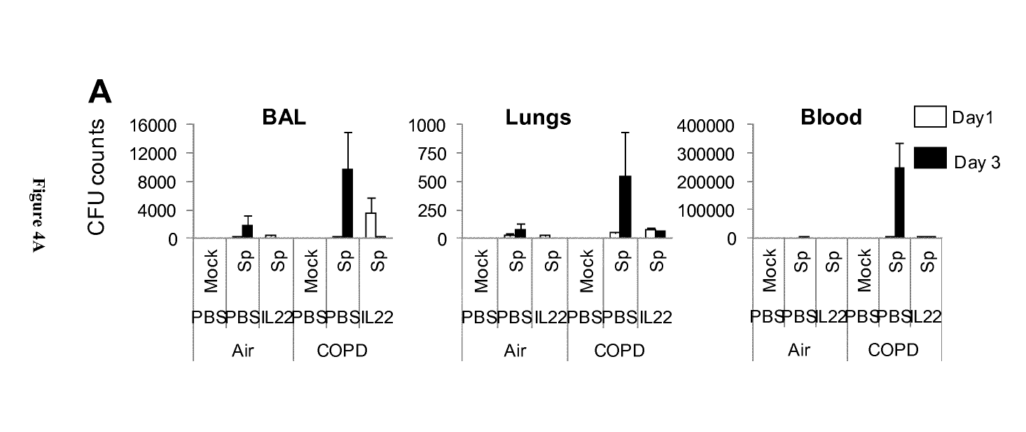

In addition to the recruitment of neutrophils, infection with Sp enhanced the number of T, NK and iNKT cells within the lung tissue of Air mice and their activation as attested by the increased expression of CD69 (FIG. 3A). Inflammation due to Sp exposure was majored in COPD mice, as shown by the increased recruitment of CD8⁺ and CD4⁺ T cells, compared to air mice. However, Sp challenge in COPD mice failed to induce a higher recruitment and activation of NKT cells, and a greater stimulation of T cells (FIG. 3A).

Since we observed a defect in the Th17 response induced by Sp in COPD mice and a defect in immune cell activation, we next investigated the cellular sources of IL-17 and IL-22 in infected Air and COPD mice (FIG. 3B). After Sp challenge, about 30% of NK cells, 20% of iNKT cells and 50% of total T cells were IL-17⁺ in the lungs. In contrast, whereas IL-17⁺ T cells were not affected in infected COPD mice, percentages of IL-17⁺ NK and iNKT cells dropped dramatically (FIG. 3C). Percentages of IL-22⁺ NK and NKT cells also respectively decreased from 2 to 0.5%, and 5 to 0.5% in COPD mice compared to air mice. In addition, percentages of IL-17⁺ and IL-22⁺ Lin− cells were also decreased in infected COPD mice as compared to air mice. IL-22⁺ T cells were also decreased in COPD mice after SP challenge compared to air mice (from 10 down to 0.5%).

These data suggest that the Th-17 response to Sp is defective in COPD mice, mainly through a defect in the response of innate lymphocytes.

Supplementation with Recombinant IL22 Partially Restores a Competent Immune Response in COPD Mice

In order to determine the role of the defect in IL-17 and IL-22 in the bacterial susceptibility of COPD mice, we next investigated the effect of recombinant murine IL-22 (rmIL-22) in our model. Since IL-17 was involved in COPD physiopathology, we focused on the role of IL-22 cytokine Given intranasally before Sp infection, rmIL-22 strongly improved the clearance of the bacteria in COPD mice since CFU counts were decreased in the BAL, the lungs and the blood (FIG. 4A). rmIL-22 supplementation was also associated to an increased recruitment of NK cells and iNKT cells, and activation of iNKT cells showed by the increased expression of CD69 (FIG. 4B). In contrast, rmIL-22 supplementation had no effect on neutrophil recuitment. These effects on the inflammatory cells were associated with an increased production of IL-17 and IFN-γ by restimulated pulmonary cells (FIG. 4C). Finally, rmIL-22 increased mRNA levels of anti-microbial peptides such as Defb2 and Defb3 (FIG. 4D).

Taken together, these data suggest that IL-22 could be a key cytokine involved in bacterial clearance in COPD mice, and to limit the consequences of COPD exacerbation.

Human Study

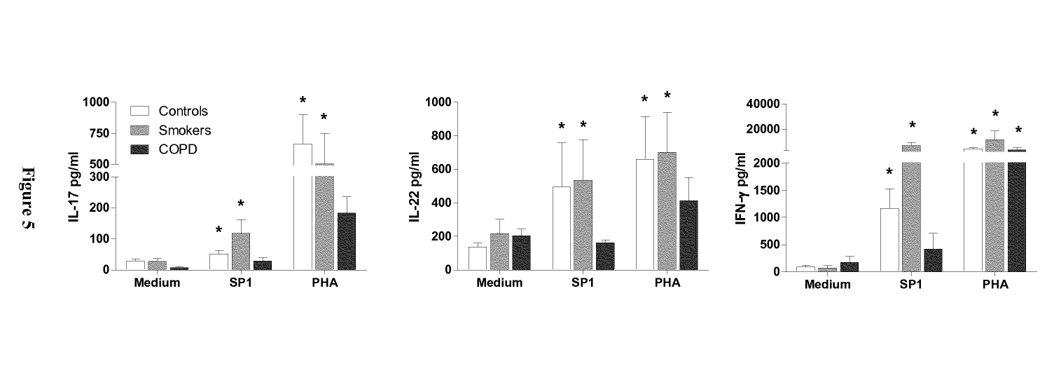

In order to evaluate the Th17 response to infection with SP1 in COPD patients, the production of these cytokines was measured in the supernatants of PBMC exposed to Sp and PHA. The concentrations of cytokines in unstimulated cells were not significantly different among the 3 groups (FIG. 5). Whereas both stimuli significantly increased the levels of IL-17, IL-22 and IFN-γ in not smokers (controls) and smokers, the exposure to Sp did not amplify the secretion of the 3 cytokines in COPD patients. The response to PHA was also partially altered in COPD patients, mainly for IL-17 and IL-22. In order to identify the cell sources for these cytokines in response to Sp and the nature of the defect in COPD patients, we analysed the intracellular staining for IL-17, IL-22 and IFN-γ in conventional T cells, NK, iNKT, Tyδ, MAIT and Lineage− (Lin−) cells. The activation with Sp significantly increased the % of IFN-γ⁺, IL-17⁺ and IL-22⁺ in innate lymphocytes (mainly NK, iNKT, MAIT and Lin− cells) from controls and COPD patients, the production of the 3 cytokines was altered in Lin− and NK cells as reported for IFN-γ and IL-22 (FIG. 5B). The production of these cytokines was also altered in iNKT cells but not in MAIT cells from COPD patients.

These data showed that the Sp-induced production of cytokines was altered in PBMC from COPD patients but not in current smokers as compared to controls. As reported in COPD mice, this defect concerns innate lymphocytes.

Conclusion:

COPD is a major public health problem and will be one of the leading global causes of mortality over the coming decades. Much of the morbidity, mortality and health care costs of COPD are attributable to acute exacerbations, the commonest causes of which are respiratory infections including SP. In this study, we develop an experimental model that accurately reflects disease pathophysiology in order to promote the development of new therapies. This study identified Th17 cytokines defect, in particular IL-22, as a key factor in COPD exacerbation in mice and humans, and could provide some insights into potential therapeutic strategies aimed at the prevention of COPD progression via normalization of the disordered innate immune mechanisms

### REFERENCES

Throughout this application, various references describe the state of the art to which this invention pertains. The disclosures of these references are hereby incorporated by reference into the present disclosure.
Aaron S D, Angel J B, Lunau M. et al Granulocyte inflammatory markers and airway infection during acute exacerbation of chronic obstructive pulmonary disease. Am J Respir Crit Care Med 2001. 163349-355.35
Agusti A G, Noguera A, Sauleda J. et al Systemic effects of chronic obstructive pulmonary disease. Eur Respir J 2003. 21347-360.3605
Aujla S J, Chan Y R, Zheng M, Fei M, Askew D J, Pociask D A, Reinhart T A, McAllister F, Edeal J, Gaus K, Husain S, Kreindler J L, Dubin P J, Pilewski J M, Myerburg M M, Mason C A, Iwakura Y, Kolls J K. 2008. IL-22 mediates mucosal host defense against Gram-negative bacterial pneumonia. Nat. Med. 14:275-281;
Barnes P J, Stockley R A., COPD: current therapeutic interventions and future approaches. Eur Respir J. 2005 June; 25(6):1084-106
Besnard A G, Sabat R, Dumoutier L, Renauld J C, Willart M, Lambrecht B, Teixeira M M, Charron S, Fick L, Erard F, Warszawska K, Wolk K, Quesniaux V, Ryffel B, Togbe D. 2011. Dual role of IL-22 in allergic airway inflammation and its cross-talk with IL-17A. Am. J. Respir. Crit. Care Med. 183:1153-1163
Caramori G, Adcock I M, Di Stefano A, Chung K F. Cytokine inhibition in the treatment of COPD. Int J Chron Obstruct Pulmon Dis. 2014 Apr. 28; 9:397-412
Clement C G, Evans S E, Evans C M, et al. Stimulation of lung innate immunity protects against lethal pneumococcal pneumonia in mice. Am J Respir Crit Care Med 2008; 177:1322-30
Colonna M. 2009. Interleukin-22-producing natural killer cells and lymphoid tissue inducer-like cells in mucosal immunity. Immunity 31:15-23
Drannik A G, Pouladi M A, Robbins C S, Goncharova S I, Kianpour S, Stampfli M R. Impact of cigarette smoke on clearance and inflammation after Pseudomonas aeruginosa infection. Am J Respir Crit Care Med. 2004; 170:1164-1171
Eyerich S, Eyerich K, Cavani A, Schmidt-Weber C. 2010. IL-17 and IL-22: siblings, not twins. Trends Immunol. 31:354-361;
Fletcher C, Peto R. The natural history of chronic airflow obstruction. Br Med J. 1977; 1:1645-1648
Gaschler G J, Bauer C M, Zavitz C C, Stampfli M R. Animal models of chronic obstructive pulmonary disease exacerbations. Contrib Microbio1.2007; 14:126-141
Gaschler G J, Skrtic M, Zavitz C C, Lindahl M, Onnervik P O, Murphy T F, Sethi S, Stampfli M R. Bacteria challenge in smoke-exposed mice exacerbates inflammation and skews the inflammatory profile. Am J Respir Crit Care Med. 2009; 179:666-675
Graham A C, Can K D, Sieve A N, Indramohan M, Break T J, Berg R E. 2011. IL-22 production is regulated by IL-23 during Listeria monocytogenes infection but is not required for bacterial clearance or tissue protection. PLoS One 6:e17171 doi:10.1371/journal.pone.0017171
Hoegl S, Bachmann M, Scheiermann P, Goren I, Hofstetter C, Pfeilschifter J, Zwissler B, Muhl H. 2011. Protective properties of inhaled IL-22 in a model of ventilator-induced lung injury. Am. J. Respir. Cell Mol. Biol. 44:369-376
Kim B H, Shenoy A R, Kumar P, Das R, Tiwari S, MacMicking J D. A family of IFN-gamma-inducible 65-kD GTPases protects against bacterial infection. Science 2011; 332:717-21;
Klugman K P, Feldman C. Streptococcus pneumoniae respiratory tract infections. Curr Opin Infect Dis 2001; 14:173-9;
Kumar P, Thakar M S, Ouyang W, Malarkannan S. 2013. IL-22 from conventional NK cells is epithelial regenerative and inflammation protective during influenza infection. Mucosal Immunol. 6:69-82
Liang S C, Tan X Y, Luxenberg D P, Karim R, Dunussi-Joannopoulos K, Collins M, Fouser L A. 2006. Interleukin (IL)-22 and IL-17 are coexpressed by Th17 cells and cooperatively enhance expression of antimicrobial peptides. J. Exp. Med. 203:2271-2279;
Lu Y J, Gross J, Bogaert D, et al. Interleukin-17A mediates acquired immunity to pneumococcal colonization. PLoS Pathog 2008; 4:e1000159
Marques J M, Rial A, Munoz N, et al. Protection against Streptococcus pneumoniae serotype 1 acute infection shows a signature of Th17- and IFN-gamma-mediated immunity. Immunobiology 2012; 78:420-29).
Martens S, Howard J. The interferon-inducible GTPases Annu Rev Cell Dev Biol 2006; 22:559-89
Mucida D, Salek-Ardakani S. Regulation of TH17 cells in the mucosal surfaces. J Allergy Clin Immunol. 2009; 123:997-1003
Munoz N, Van Maele L, Marques J M, Rial A, Sirard J C, Chabalgoity J A. Mucosal administration of flagellin protects mice from Streptococcus pneumoniae lung infection. Infect Immun 2010; 78:4226-33;
Munoz N, Van Maele L, Marques J M, Rial A, Sirard J C, Chabalgoity J A. Mucosal administration of flagellin protects mice from Streptococcus pneumoniae lung infection. Infect Immun 2010; 78:4226-33;
Paget C, Ivanov S, Fontaine J, Renneson J, Blanc F, Pichavant M, Dumoutier L, Ryffel B, Renauld J C, Gosset P, Gosset P, Si-Tahar M, Faveeuw C, Trottein F. 2012. Interleukin-22 is produced by invariant natural killer T lymphocytes during influenza A virus infection: potential role in protection against lung epithelial damages. J. Biol. Chem. 287:8816-8829
Pan H F, Li X P, Zheng S G, Ye D Q. 2013. Emerging role of interleukin-22 in autoimmune diseases. Cytokine Growth Factor Rev. 24:51-57
Rial A, Lens D, Betancor L, Benkiel H, Silva J S, Chabalgoity J A. Intranasal immunization with a colloid-formulated bacterial extract induces an acute inflammatory response in the lungs and elicits specific immune responses. Infect Immun 2004; 72:2679-88;
Sethi S, Murphy T F. Infection in the pathogenesis and course of chronic obstructive pulmonary disease. N Engl J Med. 2008; 359:2355-2365
Simonian P L, Wehrmann F, Roark C L, Born W K, O'Brien R L, Fontenot A P. 2010. γδT cells protect against lung fibrosis via IL-22. J. Exp. Med. 207:2239-2253
Soler N, Ewig S, Torres A, Filella X, Gonzalez J, Zaubet A. Airway inflammation and bronchial microbial patterns in patients with stable chronic obstructive pulmonary disease. Eur Respir J. 1999; 14:1015-1022
Soler-Cataluna J J, Martinez-Garcia M A, Roman S P. et al Severe acute exacerbations and mortality in patients with chronic obstructive pulmonary disease. Thorax 2005.60925-931.931
Sonnenberg G F, Fouser L A, Artis D. 2011. Border patrol: regulation of immunity, inflammation and tissue homeostasis at barrier surfaces by IL-22. Nat. Immunol. 12:383-390
Sonnenberg G F, Monticelli L A, Alenghat T, Fung T C, Hutnick N A, Kunisawa J, Shibata N, Grunberg S, Sinha R, Zahm A M, Tardif M R, Sathaliyawala T, Kubota M, Farber D L, Collman R G, Shaked A, Fouser L A, Weiner D B, Tessier P A, Friedman J R, Kiyono H, Bushman F D, Chang K M, Artis D. 2012 Innate lymphoid cells promote anatomical containment of lymphoid-resident commensal bacteria. Science 336:1321-1325
Sonnenberg G F, Monticelli L A, Elloso M M, Fouser L A, Artis D. 2011. CD4+ lymphoid tissue-inducer cells promote innate immunity in the gut. Immunity 34:122-134
Sonnenberg G F, Nair M G, Kim T J, Zaph C, Fouser L A, Artis D. 2010. Pathological versus protective functions of IL-22 in airway inflammation are regulated by IL-17A. J. Exp. Med. 207:1293-1305
van der Poll T, Opal S M. Pathogenesis, treatment, and prevention of pneumococcal pneumonia. Lancet 2009; 374:1543-56
Van Maele L, Carnoy C, Cayet D, Songhet P, Dumoutier L, Ferrero I, Janot L, Erard F, Bertout J, Leger H, Sebbane F, Benecke A, Renauld J C, Hardt W D, Ryffel B, Sirard J C. 2010. TLR5 signaling stimulates the innate production of IL-17 and IL-22 by CD3negCD127+ immune cells in spleen and mucosa. J. Immunol. 185:1177-1185;
Witte E, Witte K, Warszawska K, Sabat R, Wolk K. 2010. Interleukin-22: a cytokine produced by T, NK, and NKT cell subsets, with importance in the innate immune defense and tissue protection. Cytokine Growth Factor Rev. 21:365-379;
Wolk K, Kunz S, Witte E, Friedrich M, Asadullah K, Sabat R. 2004. IL-22 increases the innate immunity of tissues. Immunity 21:241-254
Zelante T, De Luca A, Bonifazi P, Montagnoli C, Bozza S, Moretti S, Belladonna M L, Vacca C, Conte C, Mosci P. et al. IL-23 and the Th17 pathway promote inflammation and impair antifungal immune resistance. Eur J Immunol. 2007; 37:2695-2706
Zemlickova H, Crisostomo M I, Brandileone M C, et al. Serotypes and clonal types of penicillin-susceptible Streptococcus pneumoniae causing invasive disease in children in five Latin American countries. Microb Drug Resist 2005; 11:195-204;
Zheng Y, Valdez P A, Danilenko D M, Hu Y, Sa S M, Gong Q, Abbas A R, Modrusan Z, Ghilardi N, de Sauvage F J, Ouyang W. 2008. Interleukin-22 mediates early host defense against attaching and effacing bacterial pathogens. Nat. Med. 14:282-289

1. A method of treating acute exacerbation of chronic obstructive pulmonary disease in a subject in need thereof comprising administering the subject with a therapeutically effective amount of an IL-22 polypeptide or an IL-17 polypeptide.

2. The method of claim 1 wherein the acute exacerbation of COPD is caused by a bacterial infection, by a viral infection or by air pollution.

3. The method of claim 2 wherein the bacterial infection is due to Streptococcus pneumoniae, Haemophilus influenzae, or Moraxella catarrhalis.

4. The method of claim 1 wherein the subject experienced an acute exacerbation of COPD or is at risk of experiencing an acute exacerbation of COPD.

5. The method of claim 1 wherein the subject is a frequent exacerbator.

6. The method of claim 1 wherein the treatment is a prophylactic treatment.

7. The method of claim 1 wherein the polypeptide is delivered to the respiratory tract.

8. The method of claim 1 wherein the polypeptide is administered to the subject in combination with an antiviral agent or an anti-bacterial agent.

9. The method of claim 8 wherein the antibacterial agent is an antibiotic.

10. The method of claim 9 wherein the antibiotic is selected from the group consisting of: ceftriaxone, cefotaxime, vancomycin, meropenem, cefepime, ceftazidime, cefuroxime, nafcillin, oxacillin, ampicillin, ticarcillin, ticarcillin/clavulinic acid (Timentin), ampicillin/sulbactam (Unasyn), azithromycin, trimethoprim-sulfamethoxazole, clindamycin, ciprofloxacin, levofloxacin, synercid, amoxicillin, amoxicillin/clavulinic acid (Augmentin), cefuroxime,trimethoprim/sulfamethoxazole, azithromycin, clindamycin, dicloxacillin, ciprofloxacin, levofloxacin, cefixime, cefpodoxime, loracarbef, cefadroxil, cefabutin, cefdinir, and cephradine.

11. The method of claim 1 wherein the polypeptide is administered to the subject in combination with at least one corticosteroid.

12. The method of claim 11 wherein the corticosteroid is selected from the group consisting of prednisolone, methylprednisolone, dexamethasone, naflocort, deflazacort, halopredone acetate, budesonide, beclomethasone dipropionate, hydrocortisone, triamcinolone acetonide, fluocinolone acetonide, fluocinonide, clocortolone pivalate, methylprednisolone aceponate, dexamethasone palmitoate, tipredane, hydrocortisone aceponate, prednicarbate, alclometasone dipropionate, halometasone, methylprednisolone suleptanate, mometasone furoate, rimexolone, prednisolone farnesylate, ciclesonide, deprodone propionate, fluticasone propionate, halobetasol propionate, loteprednol etabonate, betamethasone butyrate propionate, flunisolide, prednisone, dexamethasone sodium phosphate, triamcinolone, betamethasone 17-valerate, betamethasone, betamethasone dipropionate, hydrocortisone acetate, hydrocortisone sodium succinate, prednisolone sodium phosphate and hydrocortisone probutate.

13. The method of claim 1 wherein the administering step administers the polypeptide to the subject in combination with a bronchodilator.

14. The method of claim 13 wherein the bronchodilatator is selected from the group consisting of β2-agonists an anticholinergic, methylxanthined, and phosphodiesterase inhibitors.

15. The method of claim 1 wherein the administering step administers the polypeptide to the subject in combination with a vaccine which contains an antigen or antigenic composition capable of eliciting an immune response against a virus or a bacterium.

16. The method of claim 15 wherein the vaccine composition is used to eliciting an immune response against at least one bacterium selected from the group consisting of Streptococcus pneumoniae, Staphylococcus aureus, Burkholderis ssp., Streptococcus agalactiae, Haemophilus influenzae, Haemophilus parainfluenzae, Klebsiella pneumoniae, Escherichia coli, Pseudomonas aeruginosa, Moraxella catarrhalis, Chlamydophila pneumoniae, Mycoplasma pneumoniae, Legionella pneumophila, Serratia marcescens, Mycobacterium tuberculosis, and Bordetella pertussis.

17. The method of claim 15 wherein the vaccine composition contains whole killed or inactivated bacteria isolates.

18. The method of claim 1 wherein the polypeptide has at least 60% of identity with SEQ ID NO:1 or SEQ ID NO:2.

19. The method of claim 1 wherein the polypeptide is SEQ ID NO:1 or SEQ ID NO:2.

20. A method for the treatment of acute exacerbation of chronic obstructive pulmonary disease in a subject in need thereof comprising administering to the subject with a therapeutically effective amount of a nucleic acid molecule encoding for an IL-22 polypeptide or an IL-17 polypeptide.

21. The method of claim 14, wherein said β2-agonist is selected from the group consisting of salbutamol, bitolterol mesylate, formoterol, isoproterenol, levalbuterol, metaproterenol, salmeterol, terbutaline, and fenoterol.

22. The method of claim 14, wherein said anticholinergic is tiotropium or ipratropium.


SEQUENCE LISTING (2 sequences)

SEQ ID NO: 1 - protein, 179 aa, Homo sapiens
MAALQKSVSSFLMGTLATSCLLLLALLVQGGAAAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 2 - protein, 155 aa, Homo sapiens
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA